(19) United States
(12) Patent Application Publication
(10) Pub. No.: US 2020/0140875 A1
(43) Pub. Date: May 7, 2020

(54) GENOME EDITING METHODS FOR PRODUCING LOW-NICOTINE TOBACCO PRODUCTS

(71) Applicant: 22nd Century Limited, LLC, Williamsville, NY (US)

(72) Inventor: Paul Rushton, Williamsville, NY (US)

(73) Assignee: 22nd Century Limited, LLC, Williamsville, NY (US)

(21) Appl. No.: 16/617,951

(22) PCT Filed: May 30, 2018
(86) PCT No.: PCT/US2018/035058

§ 371 Date: Nov. 27, 2019

Related U.S. Application Data
(60) Provisional application No. 62/513,073, filed on May 31, 2017.

(51) Int. Cl. C12N 15/82; C12N 9/22; C12N 15/11

(52) U.S. Cl. CPC C12N 15/8213; C12N 9/22; C12N 2800/80; C12N 15/8243; C12N 2310/20; C12N 15/11

(57) ABSTRACT

The present technology provides targeted genome engineering (also known as genome editing) techniques to modify nicotine biosynthesis. In particular, the present technology relates to the use of genome editing methods to generate mutations resulting in an out-of-frame start codon upstream of the open reading frames (ORFs) of genes of interest, such as nicotine biosynthesis genes, to genetically engineer protein translation levels and modulate nicotine production in plants for producing plants and plant cells having reduced nicotine content.

### CROSS-REFERENCE TO RELATED APPLICATION

The present application claims priority to U.S. Application No. 62/513,073, filed on May 31, 2017, the contents of which are specifically incorporated by reference.

### TECHNICAL FIELD

The present technology relates generally to the use of targeted genome engineering (also known as genome editing) techniques to modify nicotine biosynthesis. In particular, the present technology relates to the use of genome editing methods to generate mutations resulting in an out-of-frame start codon upstream of the open reading frames (ORFs) of genes of interest, such as nicotine biosynthesis genes, to genetically engineer protein translation levels and modulate nicotine production in plants for producing plants and plant cells having reduced nicotine content.

### BACKGROUND

The following description is provided to assist the understanding of the reader. None of the information provided or references cited is admitted to be prior art.

The production of tobacco with decreased levels of nicotine is of interest given the addictiveness of nicotine and nicotine products, namely cigarettes. Additionally, tobacco plants with extremely low or no nicotine production are attractive as recipients for transgenes expressing commercially valuable products, such as pharmaceuticals, cosmetic components, or food additives. Various processes have been designed for the removal of nicotine from tobacco. However, most of these processes remove other ingredients from tobacco in addition to nicotine, thereby adversely affecting the tobacco. Classical crop breeding techniques have produced tobacco plants with lower levels of nicotine (approximately 8%) than that found in wild-type tobacco plants. Tobacco plants and tobacco having even further reductions in nicotine content are desirable.

Nicotine, a pyrrolidine alkaloid, is among the most abundant alkaloids produced in Nicotiana spp., and is synthesized in the roots and then translocates through the plant vascular system to the leaves and other aerial tissues where it serves as a defensive compound against herbivores. The enzymes involved in the major steps of nicotine biosynthetic pathway in Nicotiana tabacum (tobacco) have been characterized. Major nicotine biosynthetic pathway genes include aspartate oxidase (AO), quinolinate synthase (QS), quinolinic acid phosphoribosyltransferease (QPT), ornithine decarboxylase (ODC), arginine decarboxylase (ADC), putrescine N-methyltransferase (PMT), N-methylputrescine oxidase (MPO), diamine oxidase (DAO), an isoflavone reductase like protein, A622, and NBB1. The biosynthesis of nicotine involves pyrrolidine ring formation, pyridine ring formation, and the coupling of both rings. The enzymes arginine decarboxylase (ADC), ornithine decarboxylase (ODC), putrescine N-methyltransferase (PMT), N-methylputrescine oxidase (MPO), and diamine oxidase (DAO) are involved in the formation of the pyrrolidine ring (FIG. 1). Aspartate oxidase (AO), quinolinate synthase (QS), and quinolinic acid phosphoribosyltransferase (QPT) are enzymes responsible for the biosynthesis of the pyridine ring. These three enzymes are also involved in the synthesis of nicotinic acid dinucleotide (NAD). A622 and berberine bridge enzyme-like protein (BBL) are required for nicotine ring coupling. Data from a range of studies revealed that the regulation of nicotine biosynthesis involves a range of proteins, hormones, kinases, and transcription factors.

One approach for reducing the level of a biological product, such as nicotine, is to reduce the amount of a required enzyme in the biosynthetic pathway leading to the product. This may be accomplished by altering the expression of the gene encoding the enzyme. Although there are several known techniques for altering gene expression, including antisense technology, site-directed mutagenesis, and co-suppression, there is a need to develop precise genome targeting technologies that are affordable, scalable, amenable to targeting multiple positions within the genome, enable selective perturbation of individual genetic elements, and that can be used for the modification of tobacco nicotine levels in plants, cell lines, and derivatives thereof.

### SUMMARY

Disclosed herein are methods and compositions for reducing nicotine biosynthesis in plants. In particular, disclosed herein are targeted genome engineering (also known as genome editing) methods and compositions for altering the expression of one or more genes encoding proteins involved in the nicotine biosynthesis pathway.

In one aspect, the present disclosure provides a method for producing a targeted genomic mutation in a Nicotiana cell, the method comprising introducing into the cell at least one exogenous nuclease, wherein the nuclease cleaves endogenous genomic sequences in the cell. In some embodiments, the nuclease is selected from the group consisting of a CRISPR associated (Cas) nuclease, a meganuclease, a zinc finger protein nuclease (ZFN), a transcription activator-like effector nuclease (TALEN), and combinations thereof. In some embodiments, the targeted genomic mutation comprises an insertion, deletion, or substitution resulting in an upstream, out-of-frame start codon in a nicotine biosynthesis gene, thereby decreasing expression of a gene product of the nicotine biosynthesis gene relative to a control cell.

In some embodiments, the nicotine biosynthesis gene is selected from the group consisting of aspartate oxidase (AO), quinolinate synthase (QS), quinolinic acid phosphoribosyltransferease (QPT), ornithine decarboxylase (ODC), arginine decarboxylase (ADC), putrescine N-methyltransferase (PMT), N-methylputrescine oxidase (MPO), diamine oxidase (DAO), A622, and NBB1.

In some embodiments, the present disclosure provides a genetically engineered Nicotiana cell produced by the methods of the present technology, wherein the cell has a reduced nicotinic alkaloid content relative to a control cell.

In some embodiments, the present disclosure provides a genetically engineered Nicotiana plant comprising the genetically engineered cells, wherein the plant has a reduced nicotinic alkaloid content relative to a control plant.

In some embodiments, the present disclosure provides a product comprising the genetically engineered plant, or portions thereof, wherein the product has a reduced nicotinic alkaloid content as compared to a product produced from a control plant. In some embodiments, the product is a reduced-nicotine tobacco product selected from the group consisting of tobacco, reconstituted tobacco, cigar tobacco, pipe tobacco, cigarettes, cigars, chewing tobacco, snuff, snus, and lozenges.

In some embodiments of the methods described herein, introducing the at least one exogenous nuclease comprises introducing the nuclease as an expression construct that expresses the nuclease or as mRNA.

In one aspect, the present disclosure provides a method for reducing expression of at least one nicotine biosynthesis gene product in a Nicotiana cell comprising introducing into the cell, comprising and expressing a DNA molecule having a target sequence and encoding the gene product, an engineered CRISPR-Cas system comprising one or more vectors comprising: (a) a first regulatory element operable in a Nicotiana cell operably linked to at least one nucleotide sequence encoding a CRISPR-Cas system guide RNA that hybridizes with the target sequence, and (b) a second regulatory element operable in a Nicotiana cell operably linked to a nucleotide sequence encoding a Cas9 protein, and wherein: (i) components (a) and (b) are located on the same or different vectors of the system, (ii) the guide RNA targets the target sequence and the Cas9 protein cleaves the DNA molecule, and (iii) expression of at least one gene product is reduced relative to a control cell.

In some embodiments, the method further comprises introducing a heterologous donor oligonucleotide, wherein the heterologous donor oligonucleotide comprises a nucleotide sequence of interest to be incorporated into the genome of the Nicotiana cell. In some embodiments, the incorporation of the donor oligonucleotide into the genome of the Nicotiona cell results in an upstream, out-of-frame start codon in a nicotine biosynthesis gene, thereby decreasing expression of the gene product of the nicotine biosynthesis gene relative to a control cell.

In some embodiments, the nicotine biosynthesis gene is selected from the group consisting of aspartate oxidase (AO), quinolinate synthase (QS), quinolinic acid phosphoribosyltransferease (QPT), ornithine decarboxylase (ODC), arginine decarboxylase (ADC), putrescine N-methyltransferase (PMT), N-methylputrescine oxidase (MPO), diamine oxidase (DAO), A622, and NBB1. In some embodiments, the expression of two or more gene products is decreased.

In some embodiments, the vectors of the system further comprise one or more nuclear localization signals. In some embodiments, the guide RNAs comprise a guide sequence fused to a trans-activating cr (tracr) sequence. In some embodiments, the Cas9 protein is optimized for expression in the Nicotiana cell.

In some embodiments, the Nicotiana cell is Nicotiana tabacum.

In some embodiments, the present disclosure provides a genetically engineered Nicotiana plant comprising the cells produced by the methods described herein.

In some embodiments, the present disclosure provides a product comprising the genetically engineered plant or portions thereof, wherein the product has a reduced nicotinic alkaloid content relative to a product produced from a control plant. In some embodiments, the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges.

The technologies described and claimed herein have many attributes and embodiments including, but not limited to, those set forth or described or referenced in this brief summary. It is not intended to be all-inclusive and the inventions described and claimed herein are not limited to or by the features or embodiments identified in this brief summary, which is included for purposes of illustration only and not restriction. Additional embodiments may be disclosed in the brief description of the drawings and detailed description below.

### BRIEF DESCRIPTION OF THE DRAWINGS

FIG. 1 is a schematic diagram depicting the nicotine biosynthesis pathway. The abbreviations are: AO=aspartate oxidase, QS=quinolinate synthase, QPT=quinolinate phosphoribosyltransferase, ODC=ornithine decarboxylase, PMT=putrescine N-methyltransfrease, DAO=diamine oxidase, and BBL=berberine bridge enzyme-like protein.

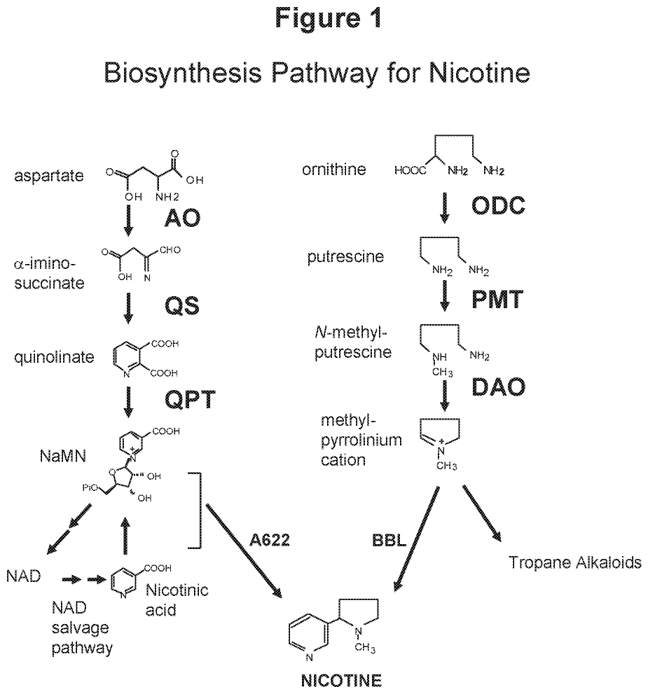

FIG. 2 is a schematic showing the effect of a genetically engineered out-of-frame upstream start codon on translation of the main open reading frame (ORF) in a gene of interest.

### DETAILED DESCRIPTION

### I. Introduction

The regulation of protein expression is a fundamental process of living cells. Many and perhaps all genes are regulated at multiple steps including transcription, post-transcriptional processing, nuclear export and localization, stability, and translation of mature mRNA molecules. Information encoded within DNA regulatory sequences controls expression output. Functional elements are also embedded within RNA sequences, such as 5′-untranslated regions (5′-UTRs). Among the various cis elements in mRNAs that participate in regulating translation are upstream AUG (uAUG) start codons, which are known to affect translation efficiency. In particular, studies have shown that out-of-frame uAUGs attenuate protein expression by acting as a barrier for downstream translation of the main open reading frame (ORF). See, e.g., Dvir et al., PNAS, E2792-E2801 (Jul. 5, 2013). Without wishing to be bound by theory, it is thought that out-of-frame uAUGs are likely to produce aberrant proteins resulting from premature termination of translation within the coding sequence of the gene of interest. When the uAUG is in-frame, a functional protein isoform with an extended N-terminal may be produced. Dvir et al. (2013). By contrast, ribosomes initiating transcription at an out-of-frame uAUG are likely to encounter an early termination codon, and this may trigger RNA degradation via nonsense-mediated decay. See, e.g., Yun et al., Genome Res., 22(6): 1089-1097 (2012). Others have shown that such genetic manipulations immediately upstream of the start codon of the main ORF (e.g., positions −10 to −1) result in considerable attenuation of protein expression. Dvir et al. (2013). In addition, it has been determined that the effect of an out-of-frame uAUG varies depending on its neighboring nucleic acid sequence. Out-of-frame uAUG variants with either purine (optimal) or pyrimidine (suboptimal) at position −3 upstream of the uAUG codon revealed an augmented reduction of protein output in the out-of-frame uAUGs with purine at the −3 position. Dvir et al. (2013).

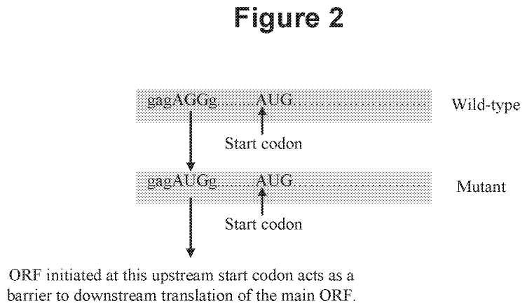

The present technology encompasses the use of targeted genome engineering (also known as genome editing) techniques that can be used to generate a mutation resulting in an out-of-frame start codon upstream of the ORFs of genes of interest to genetically engineer protein translation levels. For example, in some embodiments, the present technology contemplates the introduction of a point mutation to genetically engineer an out-of-frame start codon upstream from a gene's normal site of transcription initiation. FIG. 2 provides a schematic diagram illustrating an example in which a guanine-to-thymine mutation in the 5′-UTR of a nicotine biosynthesis gene can create an out-of-frame upstream AUG (uAUG) start codon thereby acting as a barrier for downstream translation of the main ORF of a nicotine biosynthesis gene, resulting in suppressed expression of the nicotine biosynthesis protein. In some embodiments, these precise mutations are carried out by genome editing methods provided herein. Programmable nucleases enable precise genome editing by introducing DNA double strand breaks (DSBs) at specific genomic loci. DSBs subsequently recruit endogenous repair machinery for either non-homologous end-joining (NHEJ) or homology directed repair (HDR) to the DSB site to mediate genome editing. When the DSBs are repaired by either NHEJ or HDR, the sequence at the repair site can be modified or new genetic information can be inserted (e.g., donor DNA comprising a desired mutation can be inserted into the target gene at the break site). Methods involving the use of programmable nucleases include the CRISPR (clustered regularly interspaced short palindromic repeats)/Cas (CRISPR-associated) system, meganucleases and their derivatives, zinc finger nucleases (ZFNs), and transcription activator like effector nucleases (TALENs). ZFNs, TALENs, and meganucleases achieve specific DNA binding via protein-DNA interactions. Cas9 is targeted to specific DNA sequences by a short RNA guide molecule that base-pairs directly with the target DNA.

Tobacco cells and plants modified by the methods described herein are characterized by lower nicotinic alkaloid content than control tobacco cells and plants. Tobacco plants with extremely low levels of nicotine production, or no nicotine production, are attractive as recipients for transgenes expressing commercially valuable products such as pharmaceuticals, cosmetic components, or food additives. Tobacco is attractive as a recipient plant for a transgene encoding desirable product, as tobacco is easily genetically engineered and produces a very large biomass per acre; tobacco plants with reduced resources devoted to nicotine production accordingly will have more resources available for production of transgene products. Methods of transforming tobacco with transgenes producing desired products are known in the art; any suitable technique may be utilized with the low nicotine tobacco plants of the present invention.

Tobacco plants according to the present technology with reduced expression of one or more of the nicotine biosynthesis genes and reduced nicotinic alkaloid levels will be desirable in the production of tobacco products having reduced nicotinic alkaloid content. Tobacco plants according to the present technology will be suitable for use in any tobacco product, including but not limited to chewing, pipe, cigar, cigarette tobacco, snuff, and cigarettes made from the reduced-nicotine tobacco, and may be in any form including leaf tobacco, shredded tobacco, or cut tobacco.

The genome editing techniques described herein may also be useful in providing tobacco plants having increased expression of one or more of nicotine biosynthetic pathway genes and increased nicotinic alkaloid content in the plant. Such methods and the plants so produced may be desirable in the production of tobacco products having altered nicotinic alkaloid content, or in the production of plants having nicotine content increased for its insecticidal effects.

### II. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

An “alkaloid” is a nitrogen-containing basic compound found in plants and produced by secondary metabolism. A “pyrrolidine alkaloid” is an alkaloid containing a pyrrolidine ring as part of its molecular structure, for example, nicotine. Nicotine and related alkaloids are also referred to as pyridine alkaloids in the published literature. A “pyridine alkaloid” is an alkaloid containing a pyridine ring as part of its molecular structure, for example, nicotine. A “nicotinic alkaloid” is nicotine or an alkaloid that is structurally related to nicotine and that is synthesized from a compound produced in the nicotine biosynthesis pathway. Illustrative nicotinic alkaloids include but are not limited to nicotine, nornicotine, anatabine, anabasine, anatalline, N-methylanatabine, N-methylanabasine, myosmine, anabaseine, formylnornicotine, nicotyrine, and cotinine. Other very minor nicotinic alkaloids in tobacco leaf are reported, for example, in Hecht et al., Accounts of Chemical Research 12: 92-98 (1979); Tso, T. G., Production, Physiology and Biochemistry of Tobacco Plant. Ideals Inc., Beltsville, Mo. (1990).

As used herein “alkaloid content” means the total amount of alkaloids found in a plant, for example, in terms of pg/g dry weight (DW) or ng/mg fresh weight (FW). “Nicotine content” means the total amount of nicotine found in a plant, for example, in terms of mg/g DW or FW.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant.

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. A plant is genetically engineered when a polynucleotide sequence is introduced that results in the expression of a novel gene in the plant, or an increase in the level of a gene product that is naturally found in the plants. In the present context, “genetically engineered” includes transgenic plants and plant cells, as well as plants and plant cells produced by means of chimeric repressor silencing technology (CRES-T), such as that described by Hiratsu et al., The Plant Journal 34:733-739 (2003).

“Heterologous nudeic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

Nicotine is the major alkaloid in Nicotiana tabacum and some other species in the Nicotiana genus. Other plants have nicotine-producing ability, including, for example, Duboisia, Anthoceriscis, and Salpiglossis genera in the Solanaceae, and Eclipta, and Zinnia genera in the Compositae.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores. The class of plants that can be used in the present technology is generally as broad as the class of higher plants amenable to transformation techniques, including both monocotyledonous and dicotyledonous plants. In some embodiments, the plant has a nicotine-producing capacity, such as plants of the Nicotiana, Duoisia, Anthocericis, and Salpiglossis genera in Solanaceae or the Eclipta and Zinnia genera in Compositae. In some embodiments, the plant is Nicotiana tabacum.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Decreased alkaloid plant” or “reduced alkaloid plant” encompasses a genetically engineered plant that has a decrease in alkaloid content to a level less than 50%, and preferably less than 10%, 5%, or 1% of the alkaloid content of a non-transformed control plant of the same species or variety.

“Decreased nicotine plant” or “reduced nicotine plant” encompasses a genetically engineered plant that has a decrease in nicotine content to a level less than 50%, and preferably less than 10%, 5%, or 1% of the nicotine content of a non-transformed control plant of the same species or variety.

“Increased alkaloid plant” encompasses a genetically engineered plant that has an increase in alkaloid content greater than 10⁰%, and preferably greater than 50%, 100%, or 200% of the alkaloid content of a non-transformed control plant of the same species or variety.

“Increased nicotine plant” encompasses a genetically engineered plant that has an increase in nicotine content greater than 10%, and preferably greater than 50%, 100%, or 200% of the nicotine content of a non-transformed control plant of the same species or variety.

“Loss of function” refers to the loss of function of one or more of the nicotine biosynthetic pathway genes described herein in a host tissue or organism, and encompasses the function at the molecular level (e.g., loss of transcriptional activation of downstream target genes of one or more of the transcription factors described herein), and also at the phenotypic level (e.g., reduced nicotine levels).

The terms “modification,” “genomic modification,” “modified nudeotide,” or “edited nucleotide” as used herein refer to a nucleotide sequence of interest that comprises at least one alteration when compared to its non-modified nucleotide sequence. Such “alterations” include, for example: (i) replacement or substitution of at least one nucleotide, (ii) a deletion of at least one nucleotide, (iii) an insertion of at least one nucleotide, or (iv) any combination of (i)-(iii). In some embodiments, such modifications to a gene reduce or eliminate the expression of the gene product and/or its activity.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” or “operatively linked” refers to a functional linkage between a promoter and a second sequence, where the promoter sequence initiates and mediates transcription of the DNA sequence corresponding to the second sequence. In general, “operably linked” or “operatively linked” means that the nucleic acid sequences being linked are contiguous. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame). Non-limiting examples of promoters useful in the present technology include an Arabidopsis thaliana U6 RNA polymerase III promoter, a 35S promoter, ubiquitin promoter, Rubisco small subunit promoter, an inducible promoter, including, but not limited to, an AlcR/AlcA (ethanol inducible) promoter, a glucocorticoid receptor fusion, GVG, a pOp/LhGR (dexamethasone inducible) promoter, an XCE/OlexA promoter, a heat shock promoter, and or a bidirectional promoter.

A “region of interest” is any region of cellular chromatin, such as, for example, a gene or a non-coding sequence within or adjacent to a gene, in which it is desirable to bind an exogenous molecule. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant.

As used herein, a “synergistic effect” refers to a greater-than-additive effect which is produced by a combination of at least two compounds (e.g., the effect produced by a combined overexpression of at least two dominant negative transcription factors), and which exceeds that which would otherwise result from the individual compound (e.g., the effect produced by the overexpression of a single dominant negative transcription factor alone).

“Tobacco” or “tobacco plant” refers to any species in the Nicotiana genus that produces nicotinic alkaloids, including but not limited to the following: Nicotiana acaulis, Nicotiana acuminata, Nicotiana acuminata var. multzjlora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorfi, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauczjlora, Nicotiana petunioides, Nicotiana plumbaginifolia, Nicotiana quadrivahis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegauinii, Nicotiana stocktonji, Nicotiana suaveolens, Nicotiana sylvestris, Nicotiana tabacum, Nicotiana thyrsiflora, Nicotiana tomentosa, Nicotiana tomentosifomis, Nicotiana trigonophylla, Nicotiana umbratica, Nicotiana undulata, Nicotiana velutina, Nicotiana wigandioides, and interspecific hybrids of the above.

“Tobacco product” refers to a product comprising material produced by a Nicotiana plant, including for example, cut tobacco, shredded tobacco, nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus, and lozenges.

Tobacco-specific nitrosamines (TSNAs) are a class of carcinogens that are predominantly formed in tobacco during curing, processing, and smoking. Hoffman, D., et al., J. Natl. Cancer Inst. 58:1841-4 (1977); Wiernik A et al., Recent Adv. Tob. Sci., 21:39-80 (1995). TSNAs, such as 4-(N-nitrosomethylamino)-1-(3-pyridyl)-1-butanone (NNK), N′-nitrosonornicotine (NNN), N′-nitrosoanatabine (NAT), and N′-nitrosoanabasine (NAB), are formed by N-nitrosation of nicotine and other minor Nicotiana alkaloids, such as nornicotine, anatabine, and anabasine. Reducing nicotinic alkaloids reduces the level of TSNAs in tobacco and tobacco products.

As used herein, “transformation” refers to the introduction of exogenous nucleic acid into cells, so as to produce transgenic cells stably transformed with the exogenous nucleic acid.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

As used herein, the terms “vector,” “vehicle,” “construct,” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

### III. Targeted Genome Engineering of Plants and Cells to Reduce Expression of Endogenous Nicotine Biosynthesis Genes

The present technology contemplates methods and compositions for reducing nicotine biosynthesis in plants. In particular, the present technology relates to targeted genome engineering (also known as genome editing) methods and compositions for altering the expression of one or more genes encoding proteins involved in the nicotine biosynthesis pathway. Provided herein are methods and compositions for modifying a target genomic locus in a cell to modulate the expression of one or more gene products involved in the nicotine biosynthesis pathway. Targeted genome engineering techniques described herein include the CRISPR (clustered regularly interspaced short palindromic repeats)/Cas (CRISPR-associated) system, meganucleases, zinc finger nucleases (ZFNs), and TAL effector nucleases (TALENs). Such techniques may be employed to bind to and/or cleave a region of interest upstream of the coding region of a nicotine biosynthesis gene. In some embodiments, the genome editing techniques described herein generate a specific sequence change or mutation (e.g., insertion, deletion, or substitution) in the 5′-UTR of a nicotine biosynthesis gene, such as generating a single nucleotide mutation to form an out-of-frame start codon upstream of the gene's ORF, thereby suppressing expression of the nicotine biosynthesis gene. In some embodiments, the mutation (e.g., deletion, insertion, or substitution) results in production of an upstream, out-of-frame start codon that may result in the elimination of protein production or a nonfunctional protein.

CRISPR/Cas System

In some embodiments, the methods of the present technology relate to the use of a CRISPR/Cas system that binds to a target site in a region of interest in a genome, wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering in plant cells.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Cys3, Cse1, Cse2, Csc1, Csc2, Csa5, Csn2, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcus pyogenes Cas9 protein. These enzymes are known. For example, the amino acid sequence of S. pyogenes Cas9 protein may be found in the SwissProt database under accession number Q99ZW2.

The sgRNA molecules comprise a crRNA-tacrRNA scaffold polynucleotide and a targeting sequence corresponding to a genomic target of interest.

In some embodiments, the CRISPR/Cas system recognizes a target site in a nicotine biosynthesis gene. In some embodiments, the CRISPRCas system recognizes a target in one or more of an aspartate oxidase (AO) gene, a quinolinate synthase (QS) gene, a quinolinic acid phosphoribosyltransferease (QPT) gene, an ornithine decarboxylase (ODC) gene, an arginine decarboxylase (ADC) gene, a putrescine N-methyltransferase (PMT) gene, an N-methylputrescine oxidase (MPO) gene, a diamine oxidase (DAO) gene, an A622 gene, and an NBB1 gene. The CRISPR/Cas system as described herein may bind to and/or cleave the region of interest in a region upstream of the coding region of a nicotine biosynthesis gene. In some embodiments, the CRISPR/Cas system generates a specific sequence change in the 5′-UTR of a nicotine biosynthesis gene, such as generating a single nucleotide mutation to form an out-of-frame start codon upstream of the gene's ORF.

The CRISPR/Cas system is based on the Cas9 nuclease and an engineered single guide RNA (sgRNA) that specifies the targeted nucleic acid sequence. Cas9 is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tacrRNA).

The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can induce site-specific double strand breaks (DSBs) into genomic DNA of live cells. See, e.g., Mussolino, Nat. Biothechnol., 31:208-209 (2013). In some embodiments, the Cas9 protein is expressed in a plant cell as a fusion to a nuclear localization signal (NLS) to ensure delivery into nuclei. In some embodiments, the Cas9 protein is tagged (e.g., FLAG- or GFP-tagged). In some embodiments, promoters (e.g., Cauliflower mosaic virus 35S) may be used to drive Cas9 expression in a plant cell. In some embodiments, the Cas9 enzyme is S. pneumoniae, S. pyogenes, or S. thermophiles Cas9, and may include mutated Cas9 derived from these organisms. The enzyme may be a Cas9 homolog or ortholog. In some embodiments, the CRISPR enzyme is codon-optimized for expression in a plant cell, such as a Nicotiana tabacum cell.

The single guide RNA (sgRNA) is the second component of the CRISPR/Cas system that forms a complex with the Cas9 nuclease. The sgRNA is created by fusing crRNA with tacrRNA. The sgRNA guide sequence located at the 5′ end confers DNA target specificity. By modifying the guide sequence, sgRNAs with different target specificities can be designed to target any desired endogenous gene. In some embodiments, the target sequence is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence is about 1 to about 10 base pairs upstream of the transcription start site (e.g., positions −10, −9, −8, −7, −6, −5, −4, −3, −2, or −1). It is not intended that the present technology be limited to any particular distance restraint with regard to the location of the guide RNA target sequence from the gene transcription start site. In some embodiments, the target sequence lies “in proximity to” a gene of interest, where “in proximity to” refers to any distance from the gene of interest, wherein the Cas9-regulatory domain fusion is able to exert an effect on gene expression. In some embodiments, the target sequence lies upstream of the ORF of the gene of interest.

The canonical length of the guide sequence is about 20 bp and the DNA target sequence is about 20 bp followed by a PAM sequence having the consensus NGG sequence. In some embodiments, sgRNAs are expressed in a plant cell using plant RNA polymerase III promoters, such as U6 and U3.

When the DSBs are repaired by either NHEJ or HDR, the sequence at the repair site can be modified or new genetic information can be inserted (e.g., donor DNA comprising a desired mutation can be inserted into the target gene at the break site). Although HDR typically occurs at lower and more variable frequencies than NHEJ, it can be leveraged to generate precise, defined modifications at a target locus in the presence of an exogenously introduced repair template. Accordingly, exogenous repair templates, designed by methods known in the art, can also be delivered into a cell, most often in the form of a synthetic, single-stranded DNA donor oligo or DNA donor plasmid, to generate a precise change in the genome. Single-stranded DNA donor oligos are delivered into a cell to insert or change short sequences (SNPs, amino acid substitutions, epitope tags, etc.) of DNA in the endogenous genomic target region. The benefits of using a synthetic DNA donor oligo is that no cloning is required to generate the donor template and DNA modifications can be added during synthesis for different applications, such as increased resistance to nucleases. Traditionally, the maximum insert length recommended for use with a DNA donor oligo is about 50 nucleotides.

In some embodiments, the present technology provides an engineered, programmable, non-naturally occurring CRISPR/Cas system comprising a Cas9 protein and one or more single guide RNAs (sgRNAs) that target the genomic loci of DNA molecules encoding one or more gene products in the nicotine biosynthesis pathway and the Cas9 protein cleaves the genomic loci of the DNA molecules encoding the one or more gene products, whereby expression of the one or more gene products is altered. In some embodiments, Cas9 introduces multiple DSBs in the same cell (i.e., multiplexes) via expression of one or more distinct guide RNAs.

In some embodiments, the present technology provides a method for targeted genomic modification of plant cells to alter the expression of at least one nicotine biosynthesis gene, the method comprising introducing into a plant cell, comprising and expressing a DNA molecule having a target sequence and encoding the nicotine biosynthesis gene, an engineered CRISPRCas system comprising (a) an expression construct comprising a first polynucleotide encoding a bacterial Cas9 protein, or a variant thereof or a fusion protein therewith, and a second polynucleotide encoding a guide RNA comprising: (i) a crRNA-tracrRNA scaffold polynucleotide, and (ii) a targeting sequence operably linked to the crRNA-tracrRNA scaffold polynucleotide, where the targeting sequence corresponds to a genomic locus of interest, and (b) delivering the expression construct into the plant cell, where the first and second polynucleotides are expressed (transcribed) within the plant cell. This method can optionally further include visualizing, identifying, or selecting for plant cells having a genomic modification at the genomic locus of interest that is induced by the delivering the expression construct into the plant cell.

In some embodiments of the methods of the present technology, the Cas9 polypeptide and guide RNA are encoded on two separate vectors. In these methods, the steps generally follow the sequence of introducing into a plant cell containing and expressing a DNA molecule having a target sequence and encoding the nicotine biosynthesis gene an engineered CRISPR/Cas system comprising (a) a Cas9 polynucleotide or a conservative variant thereof, and a guide RNA comprising (i) a crRNA-tracrRNA scaffold polynucleotide, and (ii) a targeting sequence operably linked to the crRNA-tracrRNA scaffold polynucleotide, with the targeting sequence corresponding to a genomic locus of interest, and (b) delivering the two polynucleotides into the plant cell. In variations of this method, a donor polynucleotide having homology to the genomic target of interest is included in a cotransfection. In some variations of these methods, the transfected material can be either plasmid DNA or RNA generated by in vitro transcription. In still other variations, the methods for targeted genomic modification are multiplexed, meaning that more than one genomic locus is targeted for modification. In still other variations of these methods, the transformation of the plant cells can be followed by visualizing, identifying, or selecting for plant cells having a genomic modification at the genomic locus of interest.

Meganucleases

In some embodiments, the compositions and methods described herein employ a meganuclease DNA binding domain for binding to a region of interest in the genome of a plant cell. Meganucleases are engineered versions of naturally occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG family, the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-Ceul, PI-Pspl, PI-Sce, I-ScelV, I-CsmlI, I-PanI, I-Scell, I-PpoI, I-SceIII, I-CreI, I-TevI, I-TevII, and I-TevIII are known.

In some embodiments, the meganuclease is tailored to recognize a target in one or more of an aspartate oxidase (AO) gene, a quinolinate synthase (QS) gene, a quinolinic acid phosphoribosyltransferease (QPT) gene, an ornithine decarboxylase (ODC) gene, an arginine decarboxylase (ADC) gene, a putrescine N-methyltransferase (PMT) gene, an N-methylputrescine oxidase (MPO) gene, a diamine oxidase (DAO) gene, an A622 gene, and an NBB1 gene. The meganucleases as described herein may bind to and/or cleave the region of interest in a region upstream of the coding region of a nicotine biosynthesis gene. Gene insertion or correction can be achieved by the introduction of a DNA repair matrix containing sequences homologous to the endogenous sequence surrounding the DNA break. Mutations can be created either at or distal to the break. In some embodiments, the meganuclease generates a specific sequence change in the 5′-UTR of a nicotine biosynthesis gene, such as generating a single nucleotide mutation to form an out-of-frame start codon upstream of the gene's ORF.

TALENs

In some embodiments, the compositions and methods described herein employ transcription activator-like effector nucleases (TALENs) to edit plant genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, after a target sequence in a nicotine biosynthesis gene is identified, a corresponding TALEN sequence is engineered and inserted into a plasmid. The plasmid is inserted into a target cell where it is translated to produce a functional TALEN, which then enters the nucleus where it binds to and cleaves its target sequence. Such an approach can be employed to introduce an exogenous DNA sequence into the target gene as the DSB is being repaired through either homology-directed repair or non-homologous end-joining. For example, in some embodiments, the use of TALEN technology generates a specific sequence change (e.g., insertion, deletion, or substitution) in the 5′-UTR of a nicotine biosynthesis gene, resulting in the production of an out-of-frame start codon upstream of the gene's ORF.

ZFNs

In some embodiments, the compositions and methods described herein employ zinc finger nucleases (ZFNs) to edit plant genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the Fold restriction enzyme responsible for cleaving DNA. ZFNs can be engineered according to methods known in the art to bind to a desired DNA sequence and cleave DNA at specific locations. For example, after a target sequence in a nicotine biosynthesis gene is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in the 5′-UTR of a nicotine biosynthesis gene, such as the insertion of an out-of-frame start codon upstream of the gene's ORF.

Quantifying Nicotinic Alkaloid Content

Methods of ascertaining nicotinic alkaloid content are available to those skilled in the art. In some embodiments of the present technology, genetically engineered plants and cells are characterized by reduced nicotinic alkaloid content. In some embodiments of the present technology, genetically engineered plants and cells are characterized by reduced nicotine content.

A quantitative reduction in nicotine levels can be assayed by several methods, as for example by quantification based on gas-liquid chromatography, high performance liquid chromatography, mass spectrometry, radio-immunoassays, and enzyme-linked immunosorbent assays.

In describing a plant of the present technology, the phrase “decreased nicotine plant” or “reduced nicotine plant” encompasses a plant that has a decrease in nicotine content to a level less than about 50%, about 40%, about 30%, about 25%, about 20%, about 15%, about 10%, about 9%, about 8%, about 7%, about 6%, about 5%, about 4%, about 3%, about 2% or about 1% of the nicotine content of a control plant of the same species or variety.

Host Plants and Cells

In some embodiments, the present technology relates to the genetic manipulation of a plant or cell via targeted genome engineering (also known as genome editing) techniques that can be used to generate a mutation resulting in an out-of-frame start codon (uAUG) immediately upstream of the ORFs of genes of interest to genetically engineer protein translation levels. In some embodiments, without wishing to be bound by theory, the introduction of an out-of-frame start codon (uAUG) attenuates protein expression by acting as a barrier for downstream translation of the main open reading frame (ORF) of a nicotine biosynthesis gene. Accordingly, the present technology provides methodology and constructs for reducing nicotine biosynthesis in a plant. Additionally, the present technology provides methods for reducing nicotine biosynthesis in a plant cell.

Plants for use in the methods of the present technology are species of Nicotiana or tobacco, including N. tabacum, N. rustica, and N. glutinosa. Any strain or variety of tobacco may be used. In some embodiments, strains that are already low in nicotine content, such as Nic1/Nic2 double mutants, are used in the methods of the present technology.

Any plant tissue capable of subsequent clonal propagation, whether by organogenesis or embryogenesis, may be transformed with a vector of the present technology. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, callus tissue, existing meristematic tissue (e.g., apical meristems, axillary buds, and root meristems), and induced meristem tissue (e.g., cotyledon meristem and hypocotyl meristem).

Plants of the present technology may take a variety of forms. The plants may be chimeras of transformed cells and non-transformed cells; the plants may be clonal transformants (e.g., all cells transformed to contain the transcription cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npi11) can be associated with the transcription cassette to assist in breeding.

In view of the foregoing, it will be apparent that plants which may be employed in practicing the present invention include those of the genus Nicotiana.

Methods of making engineered plants of the present technology, in general, involve first providing a plant cell capable of regeneration. The plant cell is then transformed with a nucleic acid construct/expression vector or other nucleic acids (such as RNA) of the present technology and an engineered plant is regenerated from the transformed plant cell. Any of the nucleic acid constructs used for reducing the expression of an endogenous nicotine biosynthetic pathway transcription factor gene can be delivered in vivo or ex vivo by any suitable means known in the art including, but not limited to, electroporation, viral transduction, viral vectors, and lentiviral vectors. In plants, expression systems have been employed to implement the CRISPR/Cas9 system. Widely used assays in plant research include protoplast transformation, the floral dip method, and leaf tissue transformation using the agroinfiltration method (also known as the Agrobacterium tumefaciens-mediated transient expression assay). See, e.g., Belhaj et al., Plant Methods, 9:39 (2013). The agroinfiltration method, which is performed on intact plants, is based on infiltration of Agrobacterium tumefaciens strains carrying a binary plasmid that contains the candidate genes to be expressed. Transgenic plants can be easily regenerated out of agroinfiltrated tissue and can be used to generate plants carrying the specified mutations. See, e.g., Nekrasov et al., Nat. Biotechnol., 31:691-693 (2013). Numerous Agrobacterium vector systems useful in methods of the present technology are known. For example, U.S. Pat. No. 4,459,355 discloses a method for transforming susceptible plants, including dicots, with an Agrobacterium strain containing the Ti plasmid. The transformation of woody plants with an Agrobacterium vector is disclosed in U.S. Pat. No. 4,795,855. Further, U.S. Pat. No. 4,940,838 discloses a binary Agrobacterium vector (i.e., one in which the Agrobacterium contains one plasmid having the vir region of a Ti plasmid but no T region, and a second plasmid having a T region but no vir region) useful in carrying out the present invention. The aforementioned methods of delivering nucleases and/or donor constructs are well known to those skilled in the art and any of the methods can be used to produce a tobacco plant having decreased expression of nicotine biosynthetic pathway transcription factors, and thus lower nicotine content than a non-transformed control tobacco plant.

After transformation of the plant cells or plant, those plant cells or plants into which the desired DNA has been incorporated may be selected by methods known in the art, including but not limited to the restriction enzyme site loss assay and the Surveyor assay. See. e.g., Belhaj et al. (2013).

Various assays may be used to determine whether the plant cell shows a change in gene expression, for example, Northern blotting or quantitative reverse transcriptase PCR (RT-PCR).

As nicotine serves as a natural pesticide that helps protect tobacco plants from damage by pests, it may be desirable to additionally transform low or no nicotine plants produced by the present methods with a transgene (such as Bacillus thuringiensis) that will confer additional insect protection.

Reduced-Nicotine Products

The methods of the present technology provide genetically-engineered cells and plants having reduced nicotine levels. For example, the present technology contemplates reducing nicotine levels through the use of targeted genome engineering techniques to generate mutations resulting in an out-of-frame start codon upstream of the ORFs of nicotine biosynthesis genes thereby suppressing protein expression in the transformed cell or plant.

As described above, tobacco plants with extremely low levels of nicotine production, or no nicotine production, are attractive as recipients for transgenes expressing commercially valuable products such as pharmaceuticals, cosmetic components, or food additives. Tobacco is attractive as a recipient plant for a transgene encoding desirable product, as tobacco is easily genetically engineered and produces a very large biomass per acre; tobacco plants with reduced resources devoted to nicotine production accordingly will have more resources available for production of transgene products.

Tobacco plants according to the present technology with reduced expression of one or more of the nicotine biosynthesis genes described herein and reduced nicotine levels will be desirable in the production of tobacco products having reduced nicotine content. Tobacco plants according to the present technology will be suitable for use in any tobacco product, including but not limited to chewing, pipe, cigar, and cigarette tobacco, snuff, and cigarettes made from the reduced-nicotine tobacco for use in smoking cessation, and may be in any form including leaf tobacco, shredded tobacco, or cut tobacco.

Because the present technology provides methods for reducing nicotinic alkaloids, tobacco-specific nitrosamines (TSNAs) may also be reduced as there is a significant, positive correlation between alkaloid content in tobacco and TSNA accumulation. For example, a significant correlation coefficient between anatabine and NAT was 0.76. See Djordjevic et al., J. Agric. Food Chem., 37:752-756 (1989). TSNAs are a class of carcinogens that are predominantly formed in tobacco during curing, processing, and smoking.

TSNAs are considered to be among the most prominent carcinogens in cigarette smoke and their carcinogenic properties are well documented. See Hecht, S. Mutat. Res., 424:127-42 (1999); Hecht, S., Toxicol., 11:559-603 (1998); Hecht, S. et al., Cancer Surv., 8:273-294 (1989). TSNAs have been cited as causes of oral cancer, esophageal cancer, pancreatic cancer, and lung cancer (Hecht & Hoffman, IARC Sci. Publ., 54-61 (1991)). In particular, TSNAs have been implicated as the causative agent in the dramatic rise of adenocarcinoma associated with cigarette smoking and lung cancer (Hoffmann et al., Crit. Rev. Toxicol., 26:199-211 (1996)).

The four TSNAs considered to be the most important by levels of exposure and carcinogenic potency and reported to be possibly carcinogenic to humans are N′-nitrosonornicotine (NNN), 4-methylnitrosoamino-1-(3-pyridyl)-1-butanone (NNK), N′-nitrosoanatabine (NAT) and N′-nitrosoanabasine (NAB) (reviewed in IARC monographs on the evaluation of the carcinogenic risk of chemical to humans, Lyon (France) Vol. 37, pp. 205-208 (1985)). These TSNAs are formed by N-nitrosation of nicotine and of the minor Nicotiana alkaloids that include nornicotine, anatabine, and anabasine.

The following levels of alkaloid compounds have been reported for mainstream smoke of non-filter cigarettes (measured in μg/cigarette): nicotine: 100-3000, nornicotine: 5-150, anatabine: 5-15, anabasine: 5-12 (Hoffmann et al., Chem. Res. Toxicol., 14:7:767-790 (2000)). Mainstream smoke of U.S. cigarettes, with or without filter tips, contain (measured in ng/cigarette): 9-180 ng NNK, 50-500 ng NNN, 3-25 ng NAB and 55-300 ng NAT. Hoffmann et al., J. Toxicol. Environ. Health, 41:1-52 (1994). It is important to note that the levels of these TSNAs in sidestream smoke are 5-10 fold above those in mainstream smoke. Hoffmann et al. (1994).

Xie et al. (2004) reported that Vector 21-41, which is a genetically-engineered reduced-nicotine tobacco produced by the down-regulation of QPT, has a total alkaloid level of about 2300 ppm, which is less than 10 percent of the wild-type tobacco. Mainstream smoke from the Vector 21-41 cigarettes had less than 10 percent of NNN, NAT, NAB, and NNK as compared to such levels of a standard full flavor cigarette produced from wild-type tobacco.

The strategy for reducing TSNAs by reducing the corresponding tobacco alkaloid precursors is currently the main focus of agricultural tobacco research. Siminszky et al., Proc. Nat. Acad. Sci. USA, 102(41) 14919-14924 (2005). Thus, to reduce formation of all TSNAs there is an urgent need to reduce the precursor nicotinic alkaloids as much as possible by genetic engineering.

Among others, U.S. Pat. Nos. 5,803,081, 6,135,121, 6,805,134, 6,907,887 and 6,959,712 and U.S. Patent Application Publication Nos. 2005/0034365 and 2005/0072047 discuss methods to reduce TSNAs.

Reduced-nicotine tobacco may also be used to produce reconstituted tobacco (Recon). Recon is produced from tobacco stems and/or smaller leaf particles by a process that closely resembles typical paper making. This process entails processing the various tobacco portions that are to be made into Recon and cutting the tobacco into a size and shape that resembles cut rag tobacco made from whole leaf tobacco. This cut recon then gets mixed with cut-rag tobacco and is ready for cigarette making.

In addition to traditional tobacco products, such as cigarette and cigar tobacco, reduced-nicotine tobacco can be used as source for protein, fiber, ethanol, and animal feeds. See U.S. Patent Application Publication No. 2002/0197688. For example, reduced-nicotine tobacco may be used as a source of Rubisco (ribulose bisphosphate carboxylase-oxygenase or fraction 1 protein) because unlike other plants, tobacco-derived Rubisco can be readily extracted in crystalline form. With the exception of slightly lower levels of methionine, Rubisco's content of essential amino acids equals or exceeds that of the FAO Provisional Pattern. Ershoff, B. H. et al., Society for Experimental Biology and Medicine, 157:626-630 (1978); Wildman, S. G. Photosynthesis Research, 73:243-250 (2002)).

For biofuels to replace a sizable portion of the world's dependence on non-renewable energy sources, co-products, such as Rubisco, are required to help defray the cost of producing this renewable energy. Greene et al., Growing Energy. How Biofuels Can End America's Oil Dependence; National Resources Defense Council (2004). Thus, the greater reduction in nicotinic alkaloids in tobacco, the greater the likelihood of a successful tobacco biomass system.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Targeted Mutagenesis Using CRISPR/Cas9 to Modulate Nicotine Biosynthesis in Nicotiana

This example demonstrates the use of a CRISPR/Cas9 system to reduce nicotine biosynthesis in a Nicotiana plant.

### Methods

Cas9 and sgRNA vectors are prepared using standard methods known in the art. See, e.g., Schiml et al., Methods in Molecular Biology, 1469: 111-122 (2016). Using sequence analysis software, the intended sgRNA targeting sequence immediately 5′ of a protospacer-adjacent motif (PAM) sequence that matches the canonical form 5′-NGG can be determined. Repair templates comprising donor DNA comprising a desired mutation in the form of either single-stranded DNA donor oligos or DNA donor plasmids are prepared according to methods known in the art. See, e.g., Ran et al., Nature Protocols, 8(11):2281-2308 (2013). In some embodiments, the donor oligos, when expressed, result in the insertion of an upstream, out-of-frame start codon. Vectors comprising Cas9 and sgRNA, and donor oligos or plasmids are transformed into Agrobacterium tumefaciens. Stable Agrobacterium-mediated transformation into N. tabacum (e.g., by floral dip transformation or agroinfiltration methods). After 10-14 days following transformation, DNA samples are extracted from plants and assayed for mutagenesis events. These CRISPR/Cas-induced mutations can be identified by, e.g., PCR/restriction enzyme assay, Surveyor nuclease assay, and/or sequencing.

Results:

It is predicted that the genetically engineered Nicotiana plants comprising cells in which the expression of one or more nicotine biosynthesis genes is suppressed by the insertion of an upstream, out-of-frame start codon will result in reduced accumulation of nicotinic alkaloids, such as nicotine, as compared with non-transformed control cell lines.

Accordingly, these results will show that targeted genome editing methods, such as CRISPR/Cas, can be used to generate a mutation resulting in an out-of-frame start codon upstream of the ORFs of nicotine biosynthesis genes to produce cells and plants with a low-nicotine phenotype.

### Example 2: The Use of Meganuclease or Other Targeted Genome Editing Methods to Produce Low Nicotine Nicotiana Plants

This example demonstrates the use of a meganuclease or other targeted genome editing methods to reduce nicotine biosynthesis in a Nicotiana plant.

The methods described in this example can be used to disrupt expression of any number of genes in the biosynthetic pathway leading to nicotine (e.g., QPT, MPO, PMT, BBL) or transcription factors that regulate the pathway (for example MYC2a, MYC2b, ERF32, ERF221/ORC1) or combinations of genes.

For example, disruption of the NtQPT2 gene and hence lowering of nicotine levels can be achieved using genome editing technologies without targeting any of the coding sequence. For example, a sequence upstream of the ATG start codon can be chosen and mutated. Progeny are then isolated where this mutation creates a new ATG just upstream of the original ATG. In two thirds of cases, this ATG will be out of frame and consequently produce a short out-of-frame non-functional peptide instead of the wild type protein because the first ATG is used by RNA polymerase H. These lines will therefore have a greatly reduced or eliminated level of QPT protein and consequently reduced levels of nicotine.

Other sequences outside of the coding region can also be targeted in order to disrupt gene expression and reduce nicotine levels. For example, the promoter region that is located upstream (5 prime) to the first ATG of the coding region can be targeted and lines chosen where expression is reduced (due to mutating the binding site for a transcription factor that is an activator of gene expression). This can be a random mutagenesis with lines chosen based on disruption of NtQPT2 gene expression, or alternatively a known binding site can be targeted. In the case of NtQPT2, it is known that the MYC2a transcription factor activates gene expression and a potential binding site in the promoter is already known. This could be targeted directly and/or other sites in the promoter could be randomly mutated. In each case, it is predicted that nicotine levels will be reduced.

Other targets outside of the coding region of the NtQPT2 gene that could be disrupted using targeted genome editing include the TATA Box (the binding site for general transcription factors including the TATA Binding Protein and the TFIID complex) and the start of transcription (where the preinitiation complex assembles). It is possible that pyramiding multiple mutations upstream of the first ATG (for example the TATA Box, MYC2a binding site, and start of transcription) will produce the greatest reductions in NtQPT2 gene expression and this can be combined with an out-of-frame upstream ATG to produce the lowest (or potentially zero) gene expression and reduced nicotine levels.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

1. A method for producing a targeted genomic mutation in a Nicotiana cell, the method comprising introducing into the cell at least one exogenous nuclease, wherein the nuclease cleaves endogenous genomic sequences in the cell.

2. The method of claim 1, wherein the nuclease is selected from the group consisting of a CRISPR associated (Cas) nuclease, a meganuclease, a zinc finger protein nuclease (ZFN), a transcription activator-like effector nuclease (TALEN), and combinations thereof.

3. The method of claim 1, wherein the targeted genomic mutation comprises an insertion, deletion, or substitution resulting in an upstream, out-of-frame start codon in a nicotine biosynthesis gene, thereby decreasing expression of a gene product of the nicotine biosynthesis gene relative to a control cell.

4. The method of claim 3, wherein the nicotine biosynthesis gene is selected from the group consisting of aspartate oxidase (AO), quinolinate synthase (QS), quinolinic acid phosphoribosyltransferease (QPT), ornithine decarboxylase (ODC), arginine decarboxylase (ADC), putrescine N-methyltransferase (PMT), N-methylputrescine oxidase (MPO), diamine oxidase (DAO), A622, and NBB1.

5. A genetically engineered Nicotiana cell produced by the method of claim 1, wherein the cell has a reduced nicotinic alkaloid content relative to a control cell.

6. A genetically engineered Nicotiana plant comprising the cells of claim 5, wherein the plant has a reduced nicotinic alkaloid content relative to a control plant.

7. A product comprising the genetically engineered plant of claim 6, or portions thereof, wherein the product has a reduced nicotinic alkaloid content as compared to a product produced from a control plant.

8. The product of claim 7, wherein the product is a reduced-nicotine tobacco product selected from the group consisting of tobacco, reconstituted tobacco, cigar tobacco, pipe tobacco, cigarettes, cigars, chewing tobacco, snuff, snus, and lozenges.

9. The method of claim 1, wherein introducing the at least one exogenous nuclease comprises introducing the nuclease as an expression construct that expresses the nuclease or as mRNA.

10. A method for reducing expression of at least one nicotine biosynthesis gene product in a Nicotiana cell comprising introducing into the cell, comprising and expressing a DNA molecule having a target sequence and encoding the gene product, an engineered CRISPR-Cas system comprising one or more vectors comprising:
(a) a first regulatory element operable in a Nicotiana cell operably linked to at least one nucleotide sequence encoding a CRISPR-Cas system guide RNA that hybridizes with the target sequence, and
(b) a second regulatory element operable in a Nicotiana cell operably linked to a nucleotide sequence encoding a Cas9 protein, and
wherein: (i) components (a) and (b) are located on the same or different vectors of the system, (ii) the guide RNA targets the target sequence and the Cas9 protein cleaves the DNA molecule, and (iii) expression of at least one gene product is reduced relative to a control cell.

11. The method of claim 10 further comprising introducing a heterologous donor oligonucleotide, wherein the heterologous donor oligonucleotide comprises a nucleotide sequence of interest to be incorporated into the genome of the Nicotiana cell.

12. The method of claim 11, wherein incorporation of the donor oligonucleotide into the genome of the Nicotiona cell results in an upstream, out-of-frame start codon in a nicotine biosynthesis gene, thereby decreasing expression of the gene product of the nicotine biosynthesis gene relative to a control cell.

13. The method of claim 10, wherein the nicotine biosynthesis gene is selected from the group consisting of aspartate oxidase (AO), quinolinate synthase (QS), quinolinic acid phosphoribosyltransferease (QPT), ornithine decarboxylase (ODC), arginine decarboxylase (ADC), putrescine N-methyltransferase (PMT), N-methylputrescine oxidase (MPO), diamine oxidase (DAO), A622, and NBB1.

14. The method of claim 10, wherein the expression of two or more gene products is decreased.

15. The method of claim 10, wherein the vectors of the system further comprise one or more nuclear localization signals.

16. The method of claim 10, wherein the guide RNAs comprise a guide sequence fused to a trans-activating cr (tracr) sequence.

17. The method of claim 10, wherein the Cas9 protein is optimized for expression in the Nicotiana cell.

18. The method of claim 10, wherein the Nicotiana cell is Nicotiana tabacum.

19. A genetically engineered Nicotiana plant comprising the cells produced by the method of claim 10.

20. A product comprising the genetically engineered plant of claim 19 or portions thereof, wherein the product has a reduced nicotinic alkaloid content relative to a product produced from a control plant.

21. The product of claim 20, wherein the product is a reduced-nicotine tobacco product selected from the group consisting of cigarette tobacco, reconstituted tobacco, cigar tobacco, pipe tobacco, cigarettes, cigars, chewing tobacco, snuff, snus, and lozenges.
